(19) United States
(12) Patent Application Publication
(10) Pub. No.: US 2009/0198113 A1
(43) Pub. Date: Aug. 6, 2009

(54) DEDICATED SPECTRAL ILLUMINATION SPECTROSCOPY

(75) Inventors: Wouter Harry Jacinth Rensen, Eindhoven (NL); Golo Von Basum, Eindhoven (NL); Markus Laubscher, Eindhoven (NL); Gerhardus Wilhelmus Lucassen, Eindhoven (NL); Miguel Palacios, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/303,431

(22) PCT Filed: Jun. 5, 2007
(86) PCT No.: PCT/IB2007/052104

§ 371 Date: Dec. 4, 2008

(30) Foreign Application Priority Data
Jun. 7, 2006 (EP) 06300565.6

(51) Int. Cl. A61B 5/1455

(52) U.S. Cl. 600/310

(57) ABSTRACT

Apparatus and method for an analyte determination in blood, relying on spectroscopic techniques, in which sample is illuminated with light having dedicated spectral characteristics. The first light source (20) is a broadband light source in the IR-range, the second light source (25) is comprised of one or more monochromatic sources, such as laser diodes. The sources are chosen to correspond to wavelength highly correlated with glucose adsorption.

### FIELD OF THE INVENTION

The present invention relates to an apparatus and method for determining an analyte level in blood, and is more particularly related to an apparatus and method for non-invasively determining the glucose level in blood.

### BACKGROUND OF THE INVENTION

The measurement of an analyte concentration in blood has some application in a wide variety of processes for diagnostics and treatments of various medical conditions. One important application is the determination of blood glucose level for persons suffering diabetes.

Diabetes is a disease related to a failure of the biological mechanisms of regulation of the glycemia, i.e. the concentration of glucose in blood. In order to help regulate the glycemia during the day and to reduce the numerous physiological problems that can occur to patients suffering diabetes—among others complicated degenerative affections which, in the eye, are especially retinopathy, metabolic affections of the uvea or cataracts—blood glucose level must be monitored as often as possible. This monitoring is essential to help determining when insulin needs to be injected, and in which quantity. Non invasive glucose sensors are therefore highly desirable, to increase the frequency of proper monitoring for patients, which won't have to use a finger prick several times a day, this operation being painful and a potential source of infections.

Different systems have been proposed to non-invasively monitor blood glucose. The systems generally rely on spectroscopic techniques, typically based on the absorption of glucose in the infrared/mid infrared region, using one or more wavelengths to irradiate a sample tissue, usually a body part such as a finger tip or ear lobe, where there are enough blood vessels and not too many skin layers. The reflected and/or transmitted light intensity is collected and analysed, and the glucose level is calculated, based on the absorbance data and the collected spectra. Such a sensor based on near infrared spectroscopy is described in U.S. Pat. No. 4,655,225, wherein blood glucose determination is performed by analysing infrared light transmitted through a finger. The light source has a range from 1000 to 2500 nm, and blood glucose level is determined using two preferred wavelengths.

However, a number of other substances have strong spectroscopic properties at the wavelengths used for sensing glucose. These molecules, such as water, proteins or fats, therefore can interfere with the measurement of glucose level, resulting in a poor selectivity due to overlapped spectral bands of all substances. Highly overlapped spectra require to measure the absorption over a broad wavelength range, and to apply multivariate calibration mathematics or regression techniques to extract glucose concentration from the measured spectrum. Due to the overlapping spectra of interfering substances, the useful signal of the analyte of interest decreases, leading to a lower accuracy of a concentration measurement of the analyte of interest.

It is difficult to establish glucose concentration from the measured spectrum, and the measurements may still suffer poor accuracy.

In one approach disclosed in document WO 2005064134, a multivariate optical element is proposed, to filter out/reduce the energy outside the band of interest. However, this method, although extracting useful signal, still suffers poor signal to noise ratio, because the number of wavelength for analysing the spectrum is reduced.

Another approach would be to increase the optical power launched into the skin. However, the total power is limited, for safety reasons on the one hand, and to conserve battery in hand-held devices on the other hand.

### SUMMARY OF THE INVENTION

It is therefore an object of the invention to provide an apparatus and method for improved selectivity and sensitivity for a spectroscopic analyte measurement, in particular for blood glucose determination.

Accordingly, the invention proposes an apparatus for determining an analyte level, the apparatus comprising:

illuminating means for illuminating a part of the body with an input spectrum, said input spectrum comprises two contributions, a first broadband contribution comprising wavelengths over a broadband range, and a second selected contribution comprising selected wavelengths correlated to the analyte absorption,

collecting means for collecting transmitted and/or reflected light,

measuring means for measuring transmitted and/or reflected light intensity as a function of wavelength,

correlating means for obtaining the analyte level responsive to transmitted and/or reflected light intensity.

Thus, an apparatus relying on spectroscopic techniques is contemplated, in which light may be absorbed by the sample, and transmitted and/or reflected light constitutes the optical signal, whose relative intensity as function of the wavelength is indicative for the compounds comprised in the sample and their concentrations. The invention proposes to irradiate a sample with a modified spectrum, and in particular by superimposing two contributions, a broadband contribution, and a second contribution with selected wavelengths.

Hence, an apparatus for determining an analyte level provided with a broadband contribution for the input light allows for a sufficient wavelength range to compensate for other substances interfering, even when there are many compounds in sample of interest, such as in blood, where it is necessary to have a maximum of useful wavelengths for the input light, in order to extract the relevant analyte information buried into other information.

Besides, since the input spectrum may further comprise a second contribution with selected wavelengths, more power at wavelengths highly correlated (positively or negatively) with the analyte absorption can be provided. In other words, the spectral regions of interest can irradiate with higher power as regions whose wavelengths are not highly correlated to the analyte absorption.

The first light contribution may be uniform over this given range.

The two light contributions may be obtained by spectrally filtering light from a white light source, thus whose spectral characteristics have been modified to match the desired spectrum, i.e. with spectral intensity higher at wavelengths highly correlated with the absorption of the given analyte (second contribution).

Alternatively, the two light contributions may also come from two different sources, a first broadband light source such as an incandescent lamp, which behaves like black body radiator, i.e., that does not show strong spectral changes over the wavelength range, or a combination of a (large) number of narrow band light sources, along with additional sources comprising wavelengths of interest, such as, for example, laser diodes or superluminous diodes.

An exemplary apparatus comprises a low-intensity broadband (white) light source covering the less important wavelengths, and a high-intensity narrowband light source at the important wavelengths.

Collecting means may comprise lenses, reflectors, collectors, and generally imaging optics, well-known in the art. Collecting means may further comprise a detector, which preferably has a uniform sensitivity in the range of wavelengths used for sensing the analyte level.

Similarly, measuring means may include a spectrometer for measuring the absorption spectrum of the reflected and/or transmitted light, or an optical wavelength analyzer. When necessary, an optoelectronic or electrical amplifier may be added, in order to amplify the detected signal, as well as all necessary electronics, signal processing tools, filtering means, to process the output spectrum, and calculate the reflected intensity as a function of wavelength.

In an exemplary embodiment, said correlating means comprises computing means to compute said analyte level according to said transmitted and/or reflected intensity and to a regression vector. The apparatus may therefore use statistical mathematics based on regression techniques to extract relevant information buried into transmitted and/or reflected light, according to techniques described below.

The apparatus may further comprise regression vector storing means for storing regression vectors. Regression vectors storing means may be comprised in the correlating means, and may comprise memory means, such as flash EEPROM, well-known in the art of signal processing, and may be reprogrammable.

Said illuminating means may be in the range of about 660 nm to about 3500 nm, more particularly in the range of about 1100 nm to about 1700 nm. One particular analyte of interest is glucose, which has strong spectroscopic properties in the IR range, and in particular at about 1680 nm.

The invention also relates to a method for non invasively determining an analyte level, comprising the steps of:
selecting wavelengths which have high correlation with the analyte absorption,
illuminating a part of a body with an input spectrum containing two contributions, a first contribution containing wavelengths over a broadband range, and a second contribution containing pre-selected wavelengths,
collecting transmitted and/or reflected light,
measuring transmitted and/or reflected light intensity as a function of wavelength,
correlating the analyte level responsive to transmitted and/or reflected light intensity.

The invention proposes to irradiate a sample with a modified spectrum, and in particular by superimposing two contributions, a broadband contribution, and a second contribution with selected wavelengths. Therefore, a good analyte detection accuracy may be achieved, while having enough wavelength range to enable compensation of other interfering substances. In a very advantageous way, a relative low amount of power is injected into the skin, for in vivo non invasive measurement.

Hence, providing a broadband contribution allows for a sufficient wavelength range to compensate for other substances interfering, even when there are many compounds in sample of interest, such as in blood, where it is necessary to have a maximum of useful wavelengths for the input light, in order to extract the relevant analyte information buried into other information.

Accordingly, the step of selecting wavelengths comprising the steps of:
illuminating reference samples with the first broadband contribution of the light,
collecting transmitted and/or reflected light,
computing a first regression vector,
selecting wavelengths for which the first regression vector has the largest amplitudes.

Because it is contemplated to add more light at important wavelengths when illuminating the sample, it is therefore desired to know what wavelength(s) is/are important for the prediction of the analyte of interest. A good way to know which wavelengths are important is a regression vector, with coefficients-amplitudes related to the analyte concentration.

A regression vector may be obtained as follows:

When it is not known which components could be in the sample, regression vectors are obtained by illuminating reference samples with a known concentration of said analyte of interest and where other substances have an unknown but varying concentration. By analysing the transmitted and/or reflected light intensity with a statistical method, the part of the spectrum representative of said analyte of interest may be found.

Samples are chosen to model all variations that could occur. For example, when it is contemplated to non-invasively measure blood glucose level, samples may have different amounts of water, of fats, of proteins, recreating artificially skin properties.

Different statistical methods such as partial least squares regression, principal component regression, artificial neural networks regression, multiple linear regression, well-known in the field of chemometrics, may be used to obtain a regression vector.

Alternatively, when the spectra of all possible components of the sample, including changes in these spectra caused by interaction between components, is known, all these spectra can be regarded as vectors. The regression vector can then be calculated by taking the inverse of that part of the vector (spectrum) of the analyte of interest that is orthogonal to all other vectors (spectra).

By obtaining a regression vector with a broadband light, it may become possible to take into account all possible components comprised in the sample, and to identify the actual wavelengths of interest for the analyte. More precisely, selected wavelengths may then correspond to wavelengths for which the regression vector has the largest amplitudes, i.e., representative of the analyte of interest.

Hence, a broadband light source may be modified to a more dedicated light source, where extra intensity is added at wavelengths where the regression vector has a large absolute value. These wavelengths could correlate positively for the analyte of interest (positive value of the regression vector), or they could correlate negatively (negative value of the regression vector). By doing so, the norm of the regression vector that corresponds to the dedicated light source is smaller than the norm of the regression vector that corresponds to the original broadband light source, and a better accuracy may thus be achieved for the analyte level determination.

Accordingly, in a preferred embodiment, the step of correlating said analyte level may further comprise the step of evaluating a second regression vector. In a very advantageous way, said second regression vector may be evaluated when reference samples are illuminated with both said first broadband contribution of the light and said selected wavelengths.

Indeed, evaluating a second regression vector may allow to take account of the modifications in the input spectrum, which may then comprise both the broadband light and the additional selected wavelengths. Hence, by providing a second regression vector corresponding to spectra obtained by irradiating samples with the entire input spectrum, it becomes possible to most accurately determine the analyte level, because both the regression vector and the analyte level are determined using the same input spectrum.

Note that the second regression vector may be obtained analytically by evaluating the expected differences, or may be once again obtained empirically and through statistical analysis.

The step of correlating said analyte level may comprise the step of computing the analyte level responsive to transmitted and/or reflected light and to said second regression vector. The analyte level can be determined by taking the inner product of the regression vector and of the transmitted and/or reflected light of the sample.

The invention also relates to a method for non invasively determining at least one analyte level as described above, using an apparatus as described above.

In an embodiment, the invention provides for a method for non invasively determining a blood glucose level as described before, using an apparatus as described above.

A good signal to noise ratio may thus be obtained, while keeping a low amount of total power launched into the sample, in particular into the skin, compared to prior art techniques where wavelengths which are not correlated to the analyte absorption are filtered out and/or, thereby keeping a useful signal but also without any possibility of noise reduction.

Note that light launched into the sample or impinging the sample may also interact with the sample, thereby generating light at a different wavelength(s), due to a scattering process, fluorescence, or a Raman process. However, the contribution of generated light is low compared to the absorption spectrum, and can therefore be neglected in the present application.

### BRIEF DESCRIPTION OF THE DRAWINGS

Other features and advantages of the present invention will become apparent from the following description of a preferred embodiment, given by way of example only, and with reference to the accompanying drawings, wherein:

FIG. 1 is a functional diagram of a method for determining an analyte level, according to the present invention,

FIG. 2 shows different input spectra with corresponding regression vectors,

FIG. 3 is a schematic view of an apparatus for determining blood glucose level, according to the present invention.

In the figures, identical numerical references refer to similar components.

### DETAILED DESCRIPTION

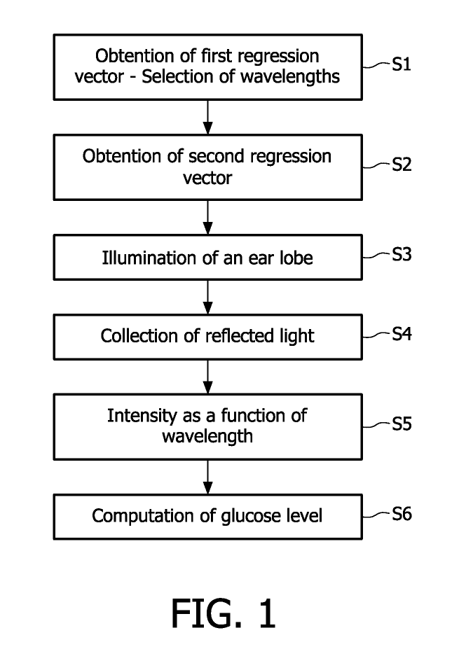

A method for determining an analyte level is described FIG. 1.

Although the method is described for glucose determination, it could be applied to various analytes, such as cholesterol (HDL and LDL), urea, uric acid, triglycerides, albumin, billirubin, and others, having an optical signature.

In the first step S1, wavelengths are selected, which have a high correlation with glucose absorption, or most generally to the analyte of interest.

This step consists in illuminating reference samples with a broadband light, in order to take account of all substances that may interfere with glucose measurements. Broadband light can be provided by an incandescent lamp that does not show strong spectral variations over the used wavelength range.

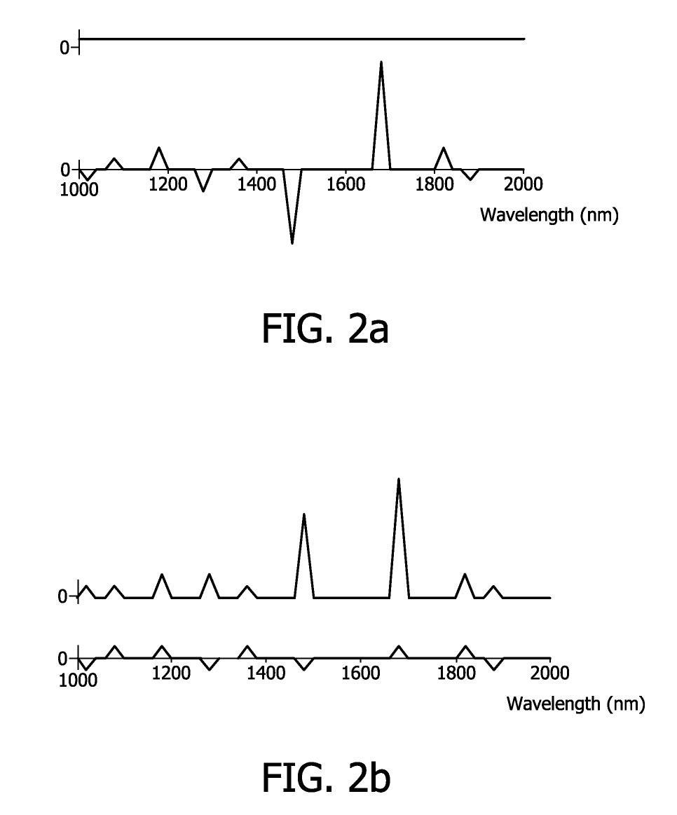

Reference samples are chosen to model all variations that can occur during the measurement. Particularly, they have a known concentration of glucose (glucose being the analyte of interest in the preferred embodiment), and where other substances (water, fats, proteins) have an unknown but varying concentration. The statistical analysis of the transmitted and/or reflected light intensity leads to a first regression vector, having different amplitudes for each wavelength, wherein the largest amplitudes represent the part of the spectrum representative of glucose. Those wavelengths, which can correlate positively (positive value of the regression vector) or negatively (negative value of the regression vector), are selected as the wavelengths highly correlated with glucose absorption. FIG. 2a depicts such a broadband input spectrum along with the corresponding first regression vector.

In a second step S2, a second regression vector is determined, this second regression vector being later used for glucose level determination, in step S6. Indeed, input spectrum used to sense glucose level is modified, and comprises two contributions, a first broadband contribution corresponding to the incandescent lamp used in step S1, and a second contribution containing wavelengths selected in the first step S1. The second regression vector should be determined, for the dedicated input spectrum.

The second regression vector may either be determined empirically, basically as described in step S1, but with the modified spectrum. Statistical analysis will lead to the regression vector. Or the second regression vector may be evaluated, based on the light spectral changes and the expected influences and variations on reflected and/or transmitted light. Examples of second regression vectors are shown on FIGS. 2b and 2c.

In a third step S3, a part of a body is illuminated with an input spectrum containing two contributions, a first contribution containing wavelengths over a broadband range, and a second contribution containing wavelengths selected in the first step S1. That is, the input spectrum is modified to match the glucose spectral characteristics, and more light is launched at wavelengths highly correlated (positively or negatively) with glucose absorption.

A preferred part of the body may be an ear lobe or a finger, where there are many blood vessels and no too many skin layers.

In step S4, reflected and/or transmitted light is collected.

In step S5, transmitted and/or reflected light intensity is measured as a function of wavelength.

In the last step S6, glucose level is computed, based on transmitted and/or reflected light intensity as a function of wavelength and on the second regression vector. More precisely, the glucose level is obtained by taking the inner product of the second regression vector and of the transmitted and/or reflected light spectrum.

S1 and S2 may be performed beforehand and the results stored in the apparatus, which comprise storing means for storing regression vectors, preferably reprogrammable.

The steps S3-S6 are performed for each measurement.

Different input spectra with corresponding regression vectors are depicted on FIG. 2a-2c.

On FIG. 2a, a broadband input spectrum and the regression vector obtained illuminating different samples comprising a known amount of a given analyte (glucose for example) with said broadband input spectrum are depicted.

Input spectrum is generally uniform over the wavelength range. Different peaks may be observed in the regression vector, positive as well as negative, corresponding to wavelengths highly correlated to the glucose absorption. A positive peak is directly linked to the analyte absorption, while a negative peak generally refers to an interfering substance.

Wavelengths where regression vector exhibits both positive and negative peaks are important, and they are selected to be the second contribution to the input spectrum for the analyte determination (corresponding to step S1 of the method illustrated on FIG. 1).

FIG. 2b shows an ideal illumination spectrum with the corresponding ideal regression vector, while FIG. 2c shows an alternative illumination spectrum with the corresponding alternative regression vector.

The ideal input spectrum comprises wavelengths selected on the basis of the regression vector obtained with said broadband spectrum (regression vector of FIG. 2a). In this case, the ideal input spectrum has a complex shape with different intensities at different wavelengths. Illuminating the reference samples with the ideal input spectrum leads to the corresponding second regression vector. All the peaks in the regression vector have the same absolute value and only change in sign.

However, it is more practical to create an illumination spectrum as shown FIG. 2c, which consists of a broadband spectrum with, in the example illustrated, two additional spectral components. In this case, the components of the second regression vector have different absolute values and different signs; however the total size (or norm) has become smaller resulting in a more accurate analyte concentration prediction.

In any case, the norm of the second regression vector corresponding to the dedicated input spectrum is smaller than the norm of the regression vector that corresponds to the original broadband spectrum, thus leading to a better accuracy of the analyte determination.

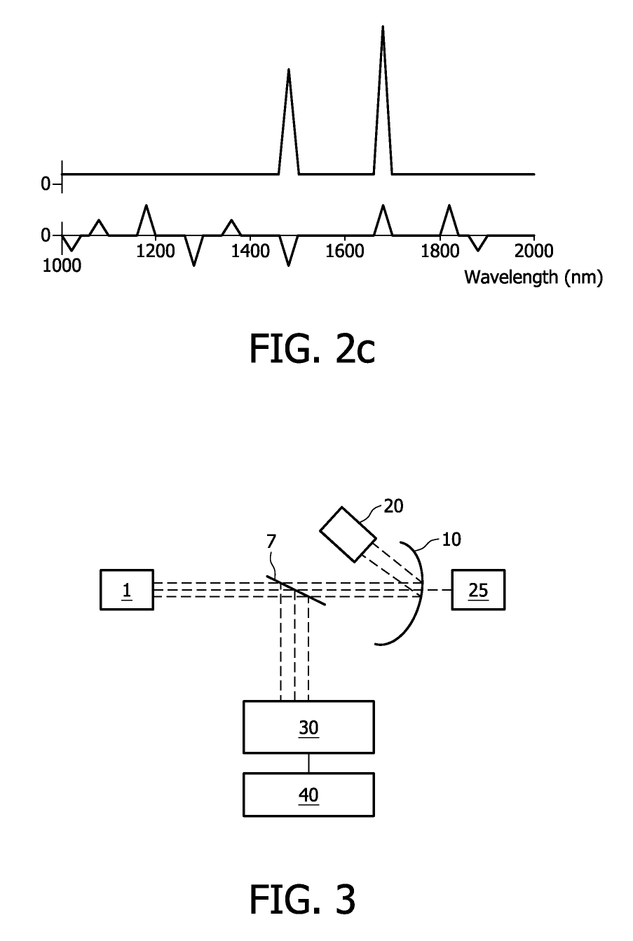

FIG. 3 is a functional diagram of an apparatus for determining an analyte level, according to the present invention.

The apparatus comprises comprise a first light source 20 and a second light source 25, which are directed towards the tissue bed, for example an ear lobe 1.

The first light source 20 is a broadband light source, in the IR range, from about 660 nm to about 3500 nm, and most preferably from about 1000 nm to about 2000 nm. Preferably, the first light source is uniform over this given range. For example, the light source may be an incandescent lamp, which behaves like black body radiator, i.e., that does not show strong spectral changes over the wavelength range.

The second light source 25 is comprised of one or more monochromatic sources, such as laser diodes, preferably of the same illumination area, with their driving electronics. The sources are chosen to correspond to wavelengths highly correlated with glucose absorption, selected as described above.

In the described embodiment, the second light source 25 comprises one or more wavelengths determined with the first regression vector, corresponding to approximately 1686 nm, the spectral peak absorption of glucose.

The apparatus further comprises a reflector 10 and imaging optics to direct the light towards the sample. In the preferred embodiment shown FIG. 2, the second light source transmits through a hole in the reflector.

The light beam from the light sources then passes through imaging optics, comprising one or more lenses for focusing the light. The light beam is then focused onto the ear lobe 1. When passing through the ear lobe 1, light is absorbed, and, in a first linear approximation, the absorbance is given by the Beer-Lambert law, wherein the absorbance at a given wavelength is given by:

A_{λ,i}=e_{λ,i}·cᵢ·lᵢ

where eᵢ is the absorptivity of a component i at wavelength λ, cᵢ is the concentration of component i, and l is the light path-length.

The light reflected off the ear lobe is redirected towards the light sources. However, a beam splitter 7 sends the reflected light towards a detector 30. The detector preferably has a uniform sensitivity in the range of wavelengths used for sensing glucose, i.e., 1000-2000 nm, and includes a spectrometer for measuring the absorption spectrum of the reflected light, or an optical wavelength analyzer. When necessary, an optoelectronic or electrical amplifier may be added, in order to amplify the detected signal.

Glucose level is then determined through the use of correlator 40, including filters and signal processing tools, computing means and memory for storing regression vectors.

More precisely, glucose level is determined by taking the inner product of the second regression vector and of the reflected light spectrum. Preferably, the second regression vector has been determined beforehand, and is stored in the memory of the apparatus.

The man skilled in the art would also recognize that this apparatus may be used to determine different analytes. In that case, different measurements can be performed, with selected wavelengths for each analyte. Another option is to use different monochromatic sources along with a multiplexer and to perform a single measurement, as it is the case for broadband light source. Tunable sources may further be employed.

Of course, for each analyte, the second selected contribution with specific wavelengths correlated to the absorption of the given analyte, must be determined, and, a second regression vector must be determined. A regression matrix, having each column corresponding to a respective analyte, may then be obtained and store in the apparatus memory for later use.

The apparatus may also comprise display and memory for storing said analyte level data. This feature may be useful for monitoring an analyte level value in time, such as blood glucose level.

The analyte sensing may be performed without harm and as often as necessary, since the techniques is non-invasive.

Thus, an apparatus and a method for determining an analyte level is disclosed, relying on spectroscopic techniques, in which light may be absorbed by the sample, and transmitted and/or reflected light constitutes the optical signal, whose relative intensity as function of the wavelength is indicative for the compounds comprised in the sample and their concentrations. By modifying the input spectrum, and in particular by superimposing two contributions, a broadband contribution, and a second contribution with selected wavelengths, a good analyte detection accuracy is achieved, while having enough wavelength range to enable compensation of other interfering substances. In a very advantageous way, a relative low amount of power injected into the skin allows for in vivo non invasive measurement.

1. Apparatus for determining an analyte level, the apparatus comprising:
an illuminating arrangement (20, 25) for illuminating a part of the body (1) with an input spectrum, said input spectrum comprises two contributions, a first broadband contribution (20) comprising wavelengths over a broadband range, and a second selected contribution (25) comprising selected wavelengths correlated to the analyte absorption,
a collector (30) for collecting transmitted and/or reflected light,
a measuring arrangement (30) for measuring transmitted and/or reflected light intensity as a function of wavelength,
a correlator (40) for obtaining the analyte level responsive to transmitted and/or reflected light intensity.

2. Apparatus according to claim 1, the correlator comprising a processing unit to compute said analyte level according to said transmitted and/or reflected intensity and to a regression vector.

3. Apparatus according to claim 2, further comprising a storage unit for storing regression vectors.

4. Apparatus according to anyone of claim 1, said illuminating arrangement being in the range of about 660 nm to about 3500 nm.

5. Method for non invasively determining an analyte level, comprising:
illuminating a part of a body with an input spectrum containing two contributions, a first broadband contribution containing wavelengths over a broadband range, and a second selected contribution containing pre-selected wavelengths,
collecting transmitted and/or reflected light,
measuring transmitted and/or reflected intensity as a function of wavelength,
correlating the analyte level responsive to transmitted and/or reflected light intensity.

6. Method according to claim 5, further comprising the step of selecting wavelengths of said second contribution.

7. Method according to claim 5, the step of selecting wavelengths comprising:
illuminating reference samples with the first broadband contribution of the light;
collecting transmitted and/or reflected light;
computing a first regression vector; and
selecting wavelengths for which the first regression vector has the largest amplitudes.

8. Method according to anyone of claims 5, further comprising the step of evaluating a second regression vector.

9. Method according to claim 8, said second regression vector being evaluated when reference samples are illuminated with both said first broadband contribution of the light and said selected wavelengths.

10. Method according to claim 8, the step of correlating said analyte level comprising the step of computing the analyte level according to transmitted and/or reflected light and to said second regression vector.
